(19) United States
(12) Patent Application Publication
(10) Pub. No.: US 2017/0248673 A1
(43) Pub. Date: Aug. 31, 2017

(54) METHOD AND APPARATUS FOR VERIFYING A PULSE SEQUENCE OF MAGNETIC RESONANCE IMAGING APPARATUS

(71) Applicant: SAMSUNG ELECTRONICS CO., LTD., Suwon-si (KR)

(72) Inventors: Jeong-a KANG, Suwon-si (KR); Sung-hun PARK, Seoul (KR); Dae-hwan KIM, Suwon-si (KR); Sung-pil JUNG, Yongin-si (KR)

(73) Assignee: SAMSUNG ELECTRONICS CO., LTD., Suwon-si (KR)

(21) Appl. No.: 15/503,788

(22) PCT Filed: Aug. 7, 2015
(86) PCT No.: PCT/KR2015/008285

§ 371 Date: Feb. 14, 2017

(30) Foreign Application Priority Data
Aug. 14, 2014 (KR) 10-2014-0106230

(51) Int. Cl. G01R 33/58; G01R 33/54

(52) U.S. Cl. CPC G01R 33/583; G01R 33/543; A61B 5/055

(57) ABSTRACT

A pulse verifying apparatus including a user input unit, which obtains a set value of a parameter for determining a magnetic resonance imaging (MRI) pulse sequence; a control unit, which compares the set value of the parameter to a critical value of the parameter and, based on a result of the comparison, determines whether an error occurred with respect to the parameter; and a display unit, which, if an error occurred with respect to the parameter, displays information regarding the error on a pulse sequence diagram corresponding to a MRI pulse sequence generated based on the set value of the parameter.

### TECHNICAL FIELD

One or more exemplary embodiments relate to a method and apparatus for verifying a pulse sequence of a magnetic resonance imaging apparatus.

### BACKGROUND ART

A magnetic resonance imaging (MRI) apparatus is an apparatus for acquiring a sectional image of a part of an object by expressing, in a contrast comparison, a strength of a MR signal with respect to a radio frequency (RF) signal generated in a magnetic field having a specific strength. For example, if an RF signal that only resonates a specific atomic nucleus (for example, a hydrogen atomic nucleus) is emitted for an instant toward the object placed in a strong magnetic field and then such emission stops, an MR signal is emitted from the specific atomic nucleus, and thus the MRI apparatus may receive the MR signal and acquire an MR image.

However, in the case of generating a magnetic field exceeding a permissible level, problems may occur in a MRI apparatus itself. Furthermore, the magnitude of a magnetic field that may be generated by each MRI apparatus is limited. Also, if RF pulses or a gradient magnetic field exceeding a permissible level is applied to a human body, the human body may be damaged.

Generally, RF pulses and a magnetic field may be generated based on set parameter values. Therefore, it is necessary for a pulse sequence designer to check whether a MRI pulse sequence generated based on set parameters is harmful to a MRI apparatus or a target object, when designing the MRI pulse sequence.

### DISCLOSURE OF INVENTION

### Solution to Problem

Provided are various embodiments for verifying a pulse sequence of a magnetic resonance imaging (MRI) apparatus.

### BRIEF DESCRIPTION OF DRAWINGS

These and/or other aspects will become apparent and more readily appreciated from the following description of the embodiments, taken in conjunction with the accompanying drawings in which:

FIG. 1 is a diagram for describing a method that a magnetic resonance imaging (MRI) apparatus picks up a MR image, according to an embodiment;

FIG. 2 is a flowchart showing a method that the pulse verifying apparatus outputs information regarding errors of a MRI pulse sequence, according to an embodiment;

FIG. 3 is a diagram showing a method that the pulse verifying apparatus according to an embodiment displays a pulse sequence diagram;

FIG. 4 is a diagram showing a method that the pulse verifying apparatus according to an embodiment displays error information by changing colors of a pulse sequence diagram;

FIG. 5 is a diagram showing a method that the pulse verifying apparatus displays error information by displaying critical values of parameters on a pulse sequence diagram, according to an embodiment;

FIG. 6A is a flowchart showing a method of displaying error information regarding a MRI pulse sequence based on characteristic values of the MRI pulse sequence, according to an embodiment;

FIG. 6B is a diagram showing a method that the pulse verifying apparatus displays error information regarding a MRI pulse sequence, according to an embodiment;

FIG. 7 is a diagram showing a method that the pulse verifying apparatus outputs information regarding time points of occurrences of errors, according to an embodiment;

FIGS. 8A and 8B are diagrams showing a method that the pulse verifying apparatus outputs error information regarding parameters, according to an embodiment;

FIG. 9 is a method that the pulse verifying apparatus displays error information regarding a MRI pulse sequence, according to an embodiment;

FIG. 10 is a diagram showing a method that the pulse verifying apparatus displays a set value of a parameter with an error, according to an embodiment;

FIG. 11 is a diagram for describing a method that the pulse verifying apparatus obtains a critical value of a parameter, according to an embodiment;

FIG. 12 is a block diagram showing the pulse verifying apparatus according to an embodiment; and,

FIG. 13 is a block diagram showing the pulse verifying apparatus according to another embodiment.

### BEST MODE FOR CARRYING OUT THE INVENTION

Additional aspects will be set forth in part in the description which follows and, in part, will be apparent from the description, or may be learned by practice of the presented embodiments.

According to one or more exemplary embodiments, a pulse verifying apparatus includes a user input unit, which obtains a set value of a parameter for determining a MRI pulse sequence; a control unit, which compares the set value of the parameter to a critical value of the parameter and, based on a result of the comparison, determines whether an error occurred with respect to the parameter; and a display unit, which, if an error occurred with respect to the parameter, displays information regarding the error on a pulse sequence diagram corresponding to a MRI pulse sequence generated based on the set value of the parameter.

The user input unit receives at least one of an identification of a MRI apparatus to generate a plurality of MRI pulses according to the MRI pulse sequence and an identification of a target object to apply the plurality of MRI pulses, and the control unit obtains a critical value of the parameter based on at least one of the identification of the MRI apparatus and the identification of the target object and compares the set value of the parameter to the obtained critical value of the parameter.

The MRI pulse sequence includes a RF pulse and a gradient magnetic field pulse, and the parameter includes at least one of slew rate of the gradient magnetic field pulse, a magnitude of the gradient magnetic field pulse, and a magnitude of the RF pulse.

The control unit generates the MRI pulse sequence based on the set value of the parameter, and the display unit displays the pulse sequence diagram indicating the generated MRI pulse sequence.

The control unit determines a MRI pulse related to the parameter corresponding to the set value exceeding the critical value from among a plurality of MRI pulses and determine a location on the time axis in the pulse sequence diagram where the determined MRI pulse sequence is displayed, and the display unit displays a pre-set image on the determined location.

The control unit determine whether power of a RF pulse included in the MRI pulse sequence exceeds a pre-set critical value, and, if the power of the RF pulse exceeds the pre-set critical value, the display unit displays an image indicating an area formed between the RF pulse displayed on the time axis in the pulse sequence diagram and the time axis.

The control unit determines a location in the pulse sequence diagram corresponding to the critical value, and the display unit displays a pre-set image at the determined location.

The display unit displays the information regarding the error by changing a color of an area in which the pulse sequence diagram is displayed.

The display unit displays information regarding a parameter having the set value exceeding the critical value.

The display unit displays an edit window for modifying the set value of the parameter, the user input unit receives a user input for modifying the set value of the parameter via the edit window, and the control unit modifies the MRI pulse sequence based on the modified set value.

According to one or more exemplary embodiments, a method of verifying a pulse sequence, the method includes obtaining a set value of a parameter for determining a MRI pulse sequence; comparing the set value of the parameter to a critical value of the parameter; based on a result of the comparison, determining whether an error occurred with respect to the parameter; and, if an error occurred with respect to the parameter, displaying information regarding the error on a pulse sequence diagram corresponding to a MRI pulse sequence generated based on the set value of the parameter.

The comparing of the set value of the parameter to a critical value of the parameter includes receiving at least one of an identification of a MRI apparatus to generate a plurality of MRI pulses according to the MRI pulse sequence and an identification of a target object to apply the plurality of MRI pulses, and obtaining a critical value of the parameter based on at least one of the identification of the MRI apparatus and the identification of the target object, and comparing the set value of the parameter to the obtained critical value of the parameter.

The MRI pulse sequence includes a RF pulse and a gradient magnetic field pulse, and the parameter includes at least one of slew rate of the gradient magnetic field pulse, a magnitude of the gradient magnetic field pulse, and a magnitude of the RF pulse.

The displaying of the information regarding the error on the pulse sequence diagram includes generating the MRI pulse sequence based on the set value of the parameter; and displaying the pulse sequence diagram indicating the generated MRI pulse sequence.

The displaying of the information regarding the error on the pulse sequence diagram includes determining a MRI pulse related to the parameter corresponding to the set value exceeding the critical value from among a plurality of MRI pulses; determining a location on the time axis in the pulse sequence diagram where the determined MRI pulse sequence is displayed; and displaying a pre-set image on the determined location.

The method further includes determining whether power of a RF pulse included in the MRI pulse sequence exceeds a pre-set critical value, wherein the displaying of the information regarding the error on the pulse sequence diagram includes, if the power of the RF pulse exceeds the pre-set critical value, displaying an image indicating an area formed between the RF pulse displayed on the time axis in the pulse sequence diagram and the time axis.

The displaying of the information regarding the error on the pulse sequence diagram includes determining a location in the pulse sequence diagram corresponding to the critical value; and displaying a pre-set image at the determined location.

The displaying of the information regarding the error on the pulse sequence diagram includes displaying the information regarding the error by changing a color of an area in which the pulse sequence diagram is displayed.

The displaying of the information regarding the error on the pulse sequence diagram includes displaying information regarding a parameter having the set value exceeding the critical value.

The displaying of the information related to the parameter includes displaying an edit window for modifying the set value of the parameter, receiving a user input for modifying the set value of the parameter via the edit window, and modifying the MRI pulse sequence based on the modified set value.

### MODE FOR THE INVENTION

Hereinafter, the terms used in the specification will be briefly defined, and the embodiments will be described in detail.

The terms used in this specification are those general terms currently widely used in the art in consideration of functions regarding the present invention, but the terms may vary according to the intention of those of ordinary skill in the art, precedents, or new technology in the art. Also, some terms may be arbitrarily selected by the applicant, and in this case, the meaning of the selected terms will be described in detail in the detailed description of the present specification. Thus, the terms used in the specification should be understood not as simple names but based on the meaning of the terms and the overall description of the invention.

When a part “includes” or “comprises” an element, unless there is a particular description contrary thereto, the part can further include other elements, not excluding the other elements. In addition, terms such as “ . . . unit”, “ . . . module”, or the like refer to units that perform at least one function or operation, and the units may be implemented as hardware or software or as a combination of hardware and software.

Reference will now be made in detail to embodiments, examples of which are illustrated in the accompanying drawings. In this regard, the present embodiments may have different forms and should not be construed as being limited to the descriptions set forth herein. Furthermore, in the drawings, any illustration irrelevant to descriptions is omitted for clarity of description, and the like reference numerals denote the like components throughout the specification.

Throughout the specification, an “image” may denote multi-dimensional data composed of discrete image elements (for example, pixels in a two-dimensional image and voxels in a three-dimensional image).

Furthermore, in the present specification, an “object” may be a human, an animal, or a part of a human or animal. For example, the object may be an organ (e.g., the liver, the heart, the womb, the brain, a breast, or the abdomen), a blood vessel, or a combination thereof. Furthermore, the “object” may be a phantom. The phantom means a material having a density, an effective atomic number, and a volume that are approximately the same as those of an organism. For example, the phantom may be a spherical phantom having properties similar to the human body.

Furthermore, in the present specification, a “user” may be, but is not limited to, a medical expert, such as a medical doctor, a nurse, a medical laboratory technologist, or a technician who repairs a medical apparatus.

Furthermore, in the present specification, a “magnetic resonance image” may be an image regarding a target object, the image obtained by utilizing the nuclear magnetic resonance.

Furthermore, in the present specification, a “MRI pulse sequence” may be a series of magnetic resonance imaging (MRI) pulses successively applied to a target object in a MRI apparatus. MRI pulses may include radio frequency (RF) pulses and gradient magnetic field pulses. Therefore, a MRI pulse sequence may include a RF pulse sequence and a gradient magnetic field sequence.

Furthermore, in the present specification, a “pulse sequence diagram” may be an image showing a sequence of events occurring inside a MRI apparatus. For example, the pulse sequence schematic diagram may be a diagram showing an RF pulse, a gradient magnetic field, an MR signal, or the like according to time.

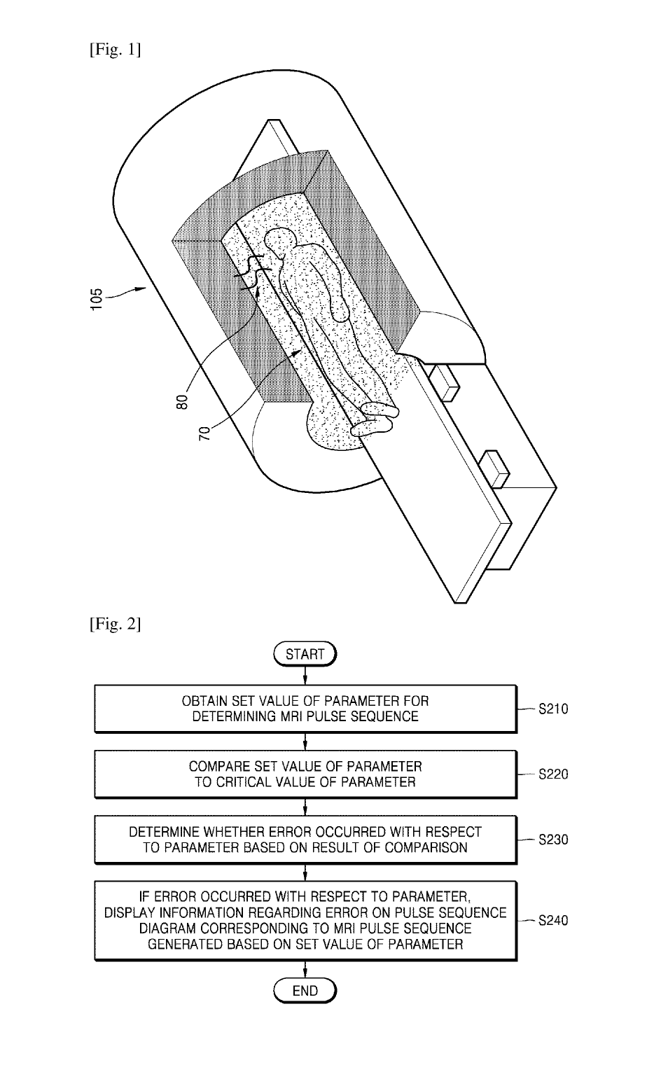

FIG. 1 is a diagram for describing a method that a MRI apparatus 105 picks up a MR image, according to an embodiment.

A user may set up a MRI pulse sequence regarding MRI pulses to be applied to a target object to pick up a MR image of the target object. A MRI pulse sequence may be set up differently based on a target object, the MRI apparatus 105, and a method of picking up a MR image. A user may develop a MRI pulse sequence by setting particular values at respective parameters regarding the MRI pulse sequence. A MRI pulse sequence may include one RF pulse sequence and three gradient magnetic field pulse sequences. The three gradient magnetic field pulse sequence may correspond to the X-axis, the Y-axis, and the Z-axis of the MRI apparatus 105, respectively.

A RF pulse sequence may include information regarding a series of RF pulses to be applied to a target object. Information regarding RF pulses may include information regarding shape, amplitude, and duration of a RF pulse and time points at which a series of RF pulses are applied to a target object.

Furthermore, a gradient magnetic field pulse sequence may include information regarding a series of gradient magnetic field pulses. Information regarding gradient magnetic field pulses may include information regarding shape, max amplitude, and duration of a gradient magnetic field pulse, an axis to apply gradient magnetic field pulses, slew rate, and time points at which gradient magnetic field pulses are applied to target object.

According to a MRI pulse sequence, the MRI apparatus 105 may apply RF pulses 80 to a target object. Furthermore, according to a MRI pulse sequence, the MRI apparatus 105 may apply a magnetic field 70 to a target object.

The RF pulses 80 and the magnetic field 70 generated by the MRI apparatus 105 and applied to a target object may cause problems at the MRI apparatus 105 or the target object.

For example, if the MRI apparatus 105 generates a gradient magnetic field exceeding a gradient or a slew rate pre-set to the MRI apparatus 105, hardware constituting the MRI apparatus 105 may malfunction. Furthermore, due to limits in hardware configuration of the MRI apparatus 105, the MRI apparatus 105 may not be able to generate a gradient magnetic field having a gradient or a slew rate exceeding a particular value.

Therefore, a permissible slew rate of a gradient magnetic field or a permissible magnitude of a gradient magnetic field may be determined based on the MRI apparatus 105. For example, a permissible slew rate of a gradient magnetic field or a permissible magnitude of a gradient magnetic field of the MRI apparatus 105 may be determined based on specification of the MRI apparatus 105 or hardware configuration of the MRI apparatus 105.

For example, maximum magnitude of gradient magnetic field pulses regarding the MRI apparatus 105 of which static magnetic field is 1.5 T (Tesla) may be decided to 33 mT/m, and maximum slew rate of the gradient magnetic field pulses may be decided to 80 T/m/s. Furthermore, maximum magnitude of gradient magnetic field pulses regarding the MRI apparatus 105 of which static magnetic field is 3.0 T may be decided to 45 mT/m, and maximum slew rate of the gradient magnetic field pulses may be decided to 200 T/m/s

Furthermore, if a magnitude or a slew rate of a gradient magnetic field pulse exceeds a critical value, the magnetic field 70 may induce peripheral nerve stimulation in a human body.

Magnitude of a gradient magnetic field pulse may indicate a gradient of a magnetic field according to a distance. Therefore, as magnitude of a gradient magnetic field pulse increases, the gradient of a magnetic field applied to a target object based on a distance may also increase. As the gradient of the magnetic field applied to the target object based on a distance increases, intensity of peripheral nerve stimulation induced in a human body may also increase.

Furthermore, a slew rate may be a rate at which a magnetic field changes according to a lapse of time. As a slew rate increases, intensity of peripheral nerve stimulation induced in a human body may increase. Furthermore, as a peripheral nerve stimulation increases, acoustic noise generated by the MRI apparatus 105 may also increase.

Furthermore, if magnitude of the RF pulses 80 or power of the RF pulses 80 exceeds a critical value, the RF pulses 80 applied to a human body may cause a burn on the human body. The power of the RF pulses 80 absorbed by a target object may be measured as a specific absorption rate (SAR). The SAR may be the power of the RF pulses 80 absorbed by a unit weight of the target object per unit time (W/kg for 1 minute average).

Effects of the magnetic field 70 and the RF pulses 80 may vary from a target object to another. For example, under the same magnetic field 70, nerve stimulation induced at an adult may be different from nerve stimulation induced at an infant. Furthermore, when the same RF pulses 80 are applied, RF power absorbed by a person having a relatively large body weight may be different from RF power absorbed by a person having a relatively small body weight.

Therefore, a permissible magnitude of gradient magnetic field pulses, a permissible slew rate of the gradient magnetic field pulses, a permissible magnitude of RF pulses, and a permissible power of the RF pulses may be determined based on a target object. For example, a permissible magnitude of gradient magnetic field pulses, a permissible slew rate of the gradient magnetic field pulses, a permissible magnitude of RF pulses, and a permissible power of the RF pulses may be determined based on age or body weight of a target object. Furthermore, a permissible magnitude of RF pulses and a permissible power of the RF pulses may be determined based on an imaged portion of the target object.

A magnitude of gradient magnetic field pulses, a slew rate of the gradient magnetic field pulses, a magnitude of RF pulses, and a power of the RF pulses may be set as parameters for determining a MRI pulse sequence. Therefore, a pulse verifying apparatus 100 may obtain parameters for determining a MRI pulse sequence and may determine whether the MRI pulse sequence may cause problems at the MRI apparatus 105 or a target object based on the obtained parameters.

Furthermore, the pulse verifying apparatus 100 may display a pulse sequence diagram showing a MRI pulse sequence based on obtained parameters. Furthermore, the pulse verifying apparatus 100 may display information regarding parameters or MRI pulses with problems in a MRI pulse sequence on a pulse sequence diagram.

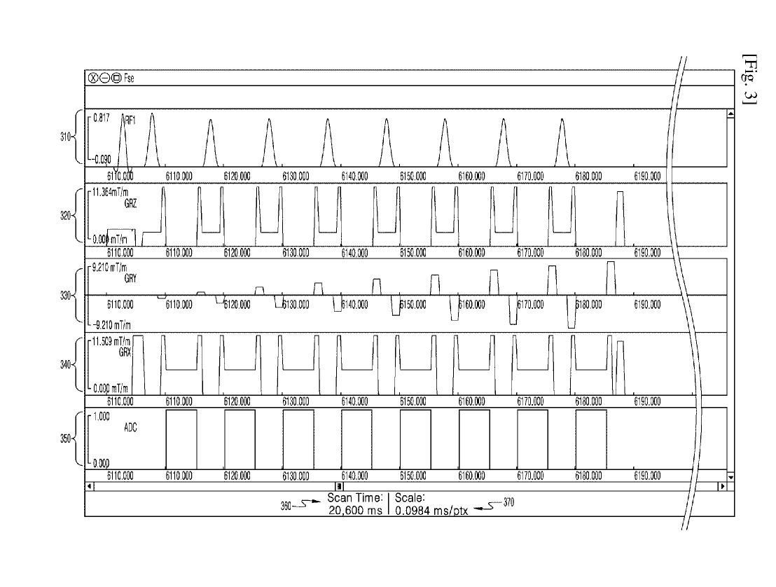

FIG. 2 is a flowchart showing a method that the pulse verifying apparatus 100 outputs information regarding errors of a MRI pulse sequence, according to an embodiment.

In an operation 5210, the pulse verifying apparatus 100 may obtain set values regarding parameters for determining a MRI pulse sequence.

The parameters for determining a MRI pulse sequence may include parameters for determining a RF pulse sequence and parameters for determining a gradient magnetic field pulse sequence. The parameters for determining a RF pulse sequence may include parameters related to shapes, magnitudes, durations, and time points of generation of a plurality of respective RF pulses. Furthermore, the parameters for determining a gradient magnetic field pulse sequence may include parameters related to shapes, slew rate, maximum magnitude, durations, and time points of generation of a plurality of respective gradient magnetic field pulses.

The pulse verifying apparatus 100 may receive a user input for inputting set values of parameters. Furthermore, the pulse verifying apparatus 100 may also receive a user input for selecting one from among a plurality of pre-determined MRI pulse sequences. Furthermore, the pulse verifying apparatus 100 may also receive a file having recorded therein set values of parameters from an external device.

In an operation 5220, the pulse verifying apparatus 100 may compare set values of parameters to critical values of parameters.

Critical values of parameters may be determined based on specification of a MRI apparatus. For example, the pulse verifying apparatus 100 may receive a user input for setting specification of a MRI apparatus and obtain critical values of parameters based on the received specification of the MRI apparatus. Specification of a MRI apparatus may include hardware configuration of the MRI apparatus and magnitude of a static magnetic field that may be generated by the MRI apparatus 105. Critical values of parameters corresponding to specification of a MRI apparatus may be stored in the pulse verifying apparatus 100 in advance.

Furthermore, critical values of parameters may be determined based on biological characteristics of a target object. For example, the pulse verifying apparatus 100 may receive a user input for setting biological characteristics of a target object and obtain critical values of parameters based on the received biological characteristics of the target object. Biological characteristics of a target object may include age, body weight, medical history, and an imaged portion of the target object. Critical values of parameters corresponding to biological characteristics of a target object may be stored in the pulse verifying apparatus 100 in advance.

Furthermore, the pulse verifying apparatus 100 may determine critical values of parameters based on at least one of identification of a MRI apparatus and identification of a target object. For example, the pulse verifying apparatus 100 may receive a user input for setting identification of a MRI apparatus. As the identification of the MRI apparatus is received, the pulse verifying apparatus 100 may obtain critical values of stored parameters based on the received identification of the MRI apparatus. Furthermore, the pulse verifying apparatus 100 may receive a user input for setting identification of a target object. As the identification of the target object is received, the pulse verifying apparatus 100 may obtain critical values of stored parameters based on the received identification of the target object.

As critical values of parameters are obtained, the pulse verifying apparatus 100 may compare set values of the parameters to the critical values of the parameter. For example, the pulse verifying apparatus 100 may determine whether a set value of a parameter exceeds the critical value of the parameter. Furthermore, the pulse verifying apparatus 100 may also determined whether a set value of a parameter is identical to or smaller than the critical value of the parameter.

In an operation 5230, the pulse verifying apparatus 100 may determine whether an error occurred with respect to a parameter based on a result of the comparison.

If a set value of a parameter exceeds the critical value of the parameter, the pulse verifying apparatus 100 may consider that an error occurred with respect to the parameter. Furthermore, if a set value of a parameter is smaller than the critical value of the parameter, the pulse verifying apparatus 100 may consider that an error occurred with respect to the parameter.

In an operation 5240, if an error occurs with respect to a parameter, the pulse verifying apparatus 100 may display information regarding the error on a pulse sequence diagram corresponding to a MRI pulse sequence generated based on a set value of the parameter.

The pulse verifying apparatus 100 may generate a MRI pulse sequence based on set values of parameter for determining a MRI pulse sequence.

For example, the pulse verifying apparatus 100 may determine a plurality of RF pulses based on shapes, magnitudes, and durations of a plurality of RF pulses and may determine the respective determined RF pulses as a single RF pulse sequence based on information regarding time points of generation of the RF pulses.

Furthermore, the pulse verifying apparatus 100 may also determine a plurality of gradient magnetic field pulses based on shapes, slew rates, magnitudes, and durations of the plurality of respective gradient magnetic field pulses and may determine the respective determined gradient magnetic field pulses as a single gradient magnetic field pulse sequence based on information regarding time points of generation of the gradient magnetic field pulses. In this case, gradient magnetic field pulse sequences may be determined with respect to the X-axis, the Y-axis, and the Z-axis, respectively.

As a MRI pulse sequence is determined, the pulse verifying apparatus 100 may display a pulse sequence diagram indicating the determined MRI pulse sequence. A pulse sequence diagram may be an image in which a plurality of MRI pulses constituting a MRI pulse sequence are shown on a time axis according to time points at which the respective MRI pulses are generated by a MRI apparatus 105. Furthermore, a pulse sequence diagram may include a user interface for displaying information regarding a MRI pulse sequence according to a user input.

The pulse verifying apparatus 100 may display error information regarding a MRI pulse sequence on a pulse sequence diagram. Error information regarding a MRI pulse sequence may include information regarding existence of a parameter with an error, information regarding a parameter with an error, and information regarding a critical value of the parameter with an error. Information regarding a parameter with an error may include information regarding a time point of generation of a MRI pulse sequence related to the parameter with the error and information regarding program codes with respect to which the parameter with the error is set.

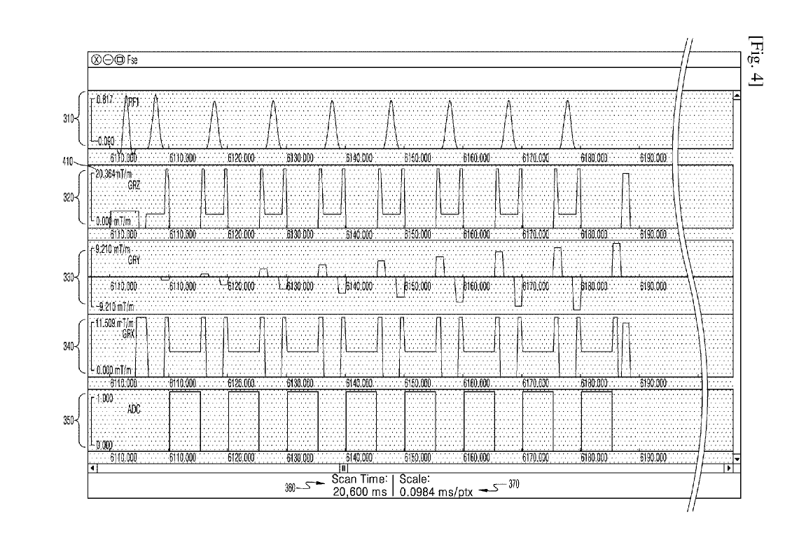

FIG. 3 is a diagram showing a method that the pulse verifying apparatus 100 according to an embodiment displays a pulse sequence diagram.

Referring to FIG. 3, the pulse verifying apparatus 100 may display a pulse sequence diagram.

The pulse verifying apparatus 100 may display a pulse sequence diagram including information regarding a RF pulse sequence 310, a Z-axis gradient magnetic field pulse sequence 320, a Y-axis gradient magnetic field pulse sequence 330, a X-axis gradient magnetic field pulse sequence 340, and an analog-to-digital conversion (ADC) sequence 350.

For example, the pulse verifying apparatus 100 may determine a plurality of RF pulses based on shapes, magnitudes, and durations of the plurality of respective RF pulses and display the respective RF pulses on a time axis based on information regarding time points of generation of the respective RF pulses. Furthermore, the pulse verifying apparatus 100 may determine a plurality of gradient magnetic field pulses based on shapes, slew rates, magnitudes, and durations of the plurality of respective gradient magnetic field pulses and display the respective gradient magnetic field pulses on a time axis based on information regarding time points of generation of the respective gradient magnetic field pulses.

In a pulse sequence diagram, a RF pulse sequence and a gradient magnetic field pulse sequence may be displayed on time axes having a same time scale.

Furthermore, the pulse verifying apparatus 100 may display overall time information 360 and time scale information 370 regarding a MRI pulse sequence on a pulse sequence diagram

Furthermore, in a pulse sequence diagram, vertical axis with respect to each pulse sequence may indicate magnitude of each pulse.

Therefore, a user may quickly check generation of RF pulses or gradient magnetic field pulses according to the lapse of time from a pulse sequence diagram.

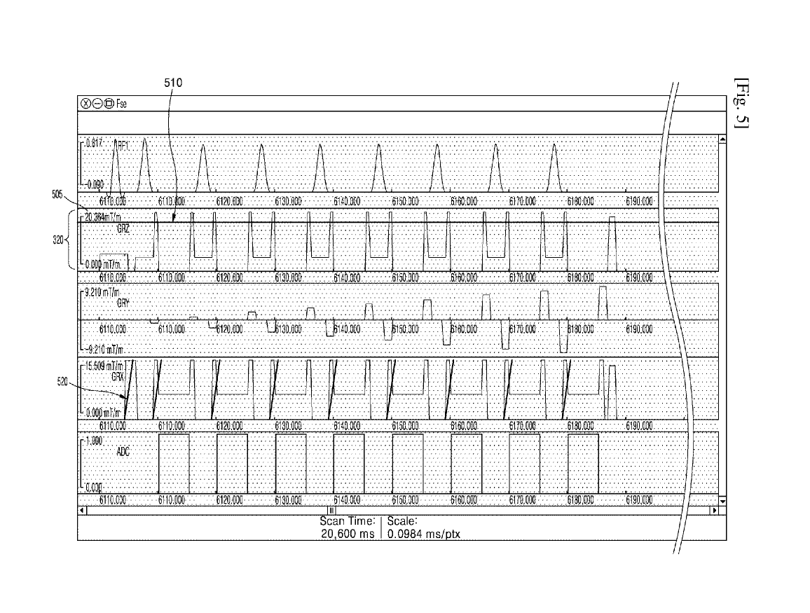

FIG. 4 is a diagram showing a method that the pulse verifying apparatus 100 according to an embodiment displays error information by changing colors of a pulse sequence diagram.

Referring to FIG. 4, the pulse verifying apparatus 100 may display error information regarding parameters by changing colors of a pulse sequence diagram.

The pulse verifying apparatus 100 may compare set values of parameters to critical values of the parameter and determine occurrence of errors with respect to the parameter. For example, if the critical value regarding magnitude of the Z-axis gradient magnetic field pulse 320 is 18 mT/m and a set value 410 regarding magnitude of the Z-axis gradient magnetic field pulse 320 is 20.36 mT/m, the set value 410 regarding magnitude of the Z-axis gradient magnetic field pulse 320 exceeds the critical value regarding magnitude of the Z-axis gradient magnetic field pulse 320, and thus the pulse verifying apparatus 100 may determined that an error occurred at magnitude of the Z-axis gradient magnetic field pulse 320.

When an error occurs at a parameter, the pulse verifying apparatus 100 may change a color of an area at which a pulse sequence diagram is displayed.

For example, if the background color in a pulse sequence diagram is white, the pulse verifying apparatus 100 may indicate occurrence of an error at magnitude of the Z-axis gradient magnetic field pulse 320 by changing the background color from white to red.

In this case, the pulse verifying apparatus 100 may change color of an entire area in which a MRI pulse sequence is displayed. Furthermore, the pulse verifying apparatus 100 may change color of only an area in which a MRI pulse sequence related to a parameter with an error is displayed. For example, the pulse verifying apparatus 100 may change color of only an area in which the Z-axis gradient magnetic field pulse sequence 320 is displayed.

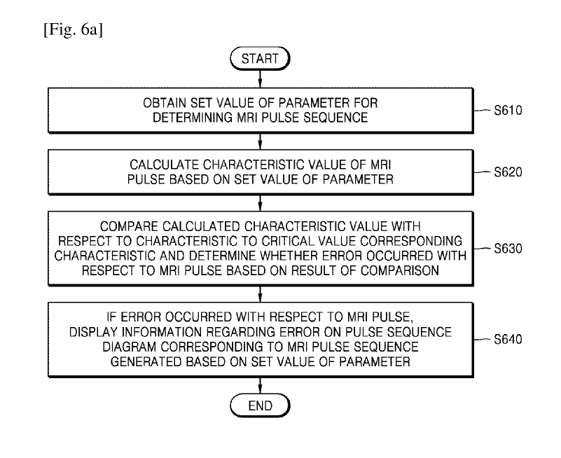

FIG. 5 is a diagram showing a method that the pulse verifying apparatus 100 displays error information by displaying critical values of parameters on a pulse sequence diagram, according to an embodiment.

Referring to FIG. 5, the pulse verifying apparatus 100 may display error information by displaying critical values of parameters on a pulse sequence diagram.

If it is determined that an error occurred with respect to a parameter, the pulse verifying apparatus 100 may determine a location in a pulse sequence diagram corresponding to the critical value corresponding the parameter and display a pre-set image at the determined location. Pre-set images may include various images, e.g., a straight line, an arrow, etc.

For example, if the critical value regarding magnitude of the Z-axis gradient magnetic field pulse sequence 320 is 18 mT/m and a set value 505 regarding the magnitude of the Z-axis gradient magnetic field pulse sequence 320 is 20.35 mT/m, the pulse verifying apparatus 100 may determine that an error occurred with respect to the magnitude of the Z-axis gradient magnetic field pulse sequence 320.

As an error occurred with respect to the magnitude of the Z-axis gradient magnetic field pulse sequence 320, the pulse verifying apparatus 100 may determine a location in a pulse sequence diagram corresponding to the critical value of the magnitude of the Z-axis gradient magnetic field pulse sequence 320. The location in the pulse sequence diagram corresponding to the critical value of the magnitude of the Z-axis gradient magnetic field pulse sequence 320 may be a location on the vertical axis indicating the magnitude of the Z-axis gradient magnetic field pulse sequence 320, which is where the magnitude of the Z-axis gradient magnetic field pulse sequence 320 is 18 mT/m.

As the location in the pulse sequence diagram corresponding to the critical value of the magnitude of the Z-axis gradient magnetic field pulse sequence 320 is determined, the pulse verifying apparatus 100 may display a straight line 510 indicating the critical value at the location where the magnitude of the Z-axis gradient magnetic field pulse sequence 320 is 18 mT/m.

Furthermore, for example, if the critical value of slew rate of the X-axis gradient magnetic field pulse sequence 340 is 50 T/m/s and a set value of the slew rate of the X-axis gradient magnetic field pulse sequence 340 is 70 T/m/s, the pulse verifying apparatus 100 may determine that an error occurred at the slew rate of the X-axis gradient magnetic field pulse sequence 340.

As an error occurred at the slew rate of the X-axis gradient magnetic field pulse sequence 340, the pulse verifying apparatus 100 may display an image 520 indicating the critical value of the slew rate of the X-axis gradient magnetic field pulse sequence 340 at an area in which the X-axis gradient magnetic field pulse sequence 340 is displayed.

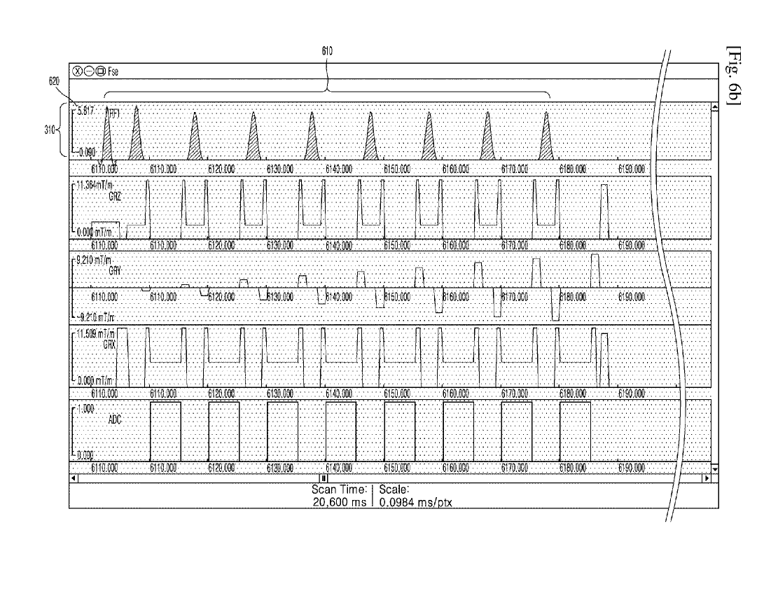

FIG. 6A is a flowchart showing a method of displaying error information regarding a MRI pulse sequence based on characteristic values of the MRI pulse sequence, according to an embodiment.

In an operation S610, the pulse verifying apparatus 100 may obtain set values of parameters for determining a MRI pulse sequence.

Parameters for determining a MRI pulse sequence may include parameters for determining a RF pulse sequence and parameters for determining a gradient magnetic field pulse sequence. The parameters for determining a RF pulse sequence may include parameters related to shapes, magnitudes, durations, and time points of generation of a plurality of respective RF pulses. Furthermore, the parameters for determining a gradient magnetic field pulse sequence may include parameters related to shapes, slew rate, maximum magnitude, durations, and time points of generation of a plurality of respective gradient magnetic field pulses.

The pulse verifying apparatus 100 may receive a user input for inputting set values of parameters. Furthermore, the pulse verifying apparatus 100 may also receive a user input for selecting one from among a plurality of pre-determined MRI pulse sequences. Furthermore, the pulse verifying apparatus 100 may also receive a file having recorded therein set values of parameters from an external device.

In an operation S620, the pulse verifying apparatus 100 may calculate characteristic values of a pulse sequence based on set values of parameters.

Characteristics of MRI pulses may include particular characteristics which affect a MRI apparatus or a target object. For example, such characteristics of MRI pulses may include power of RF pulses. As the power of RF pulses increase, a target object may get burned, and a MRI apparatus may malfunction due to heat applied thereto.

The pulse verifying apparatus 100 may calculate power regarding a plurality of RF pulses generated by a MRI apparatus based on set values of magnitudes and durations of the RF pulses from among parameters regarding the RF pulses.

Furthermore, the pulse verifying apparatus 100 may calculate not only power of RF pulses applied to a target object, but also power of RF pulses absorbed by the target object. Power of RF pulses absorbed by a target object may be proportional to the square of magnitude of a static magnetic field, the square of a flip angle, RF duty cycle, and size of a patient. Therefore, the pulse verifying apparatus 100 may also calculate power of RF pulse to be absorbed by a target object based on set values regarding magnitude of a static magnetic field, a flip angle, RF duty cycle, and size of the target object.

In an operation S630, the pulse verifying apparatus 100 may compare a calculated characteristic value with respect to a characteristic to a critical value corresponding the characteristic and may determined whether an error occurred at a MRI pulse sequence based on a result of the comparison.

A critical value of a characteristic of a MRI pulse sequence may be determined based on specification of a MRI apparatus. Specification of a MRI apparatus may include magnitude of a static magnetic field that may be generated by the MRI apparatus. Furthermore, a critical value of a characteristic of a MRI pulse sequence may be determined based on biological characteristics of a target object. Biological characteristics of a target object may include age, body weight, medical history, and imaged portion of the target object.

Furthermore, the pulse verifying apparatus 100 may determined a critical value of a characteristic of a MRI pulse sequence based on at least one of identification of a MRI apparatus and identification of a target object. As the identification of the target object is received, the pulse verifying apparatus 100 may obtain a critical value of a characteristic of a MRI pulse sequence that is stored in advance in correspondence to identification of the target object.

As the critical value of the characteristic of the MRI pulse sequence is obtained, the pulse verifying apparatus 100 may compare a set value of the characteristic of the MRI pulse sequence to the obtained critical value. Furthermore, based on a result of the comparison, it may be determined whether an error occurred at the MRI pulse sequence.

For example, as powers of a plurality of RF pulses generated by a MRI apparatus are calculated, the pulse verifying apparatus 100 may determine whether calculated power of each of the plurality of RF pulses exceeds a critical value of power of a RF pulse. If power of any of the plurality of RF pulses exceeds the critical value of power of a RF pulse, the pulse verifying apparatus 100 may determined that an error occurred with respect to RF pulses.

Furthermore, for example, as powers of RF pulses absorbed by a target object are calculated, the pulse verifying apparatus 100 may determine whether a calculated amount of the RF pulses exceeds a critical value of an amount of RF pulses to be absorbed. If a calculated amount of the RF pulses exceeds a critical value of an amount of RF pulses to be absorbed, the pulse verifying apparatus 100 may determine that an error occurred with respect to RF pulses.

In an operation S640, if an error occurred with respect to MRI pulses, the pulse verifying apparatus 100 may display information regarding the error on a pulse sequence diagram corresponding to a MRI pulse sequence generated based on set values of parameters.

The pulse verifying apparatus 100 may generate a MRI pulse sequence based on set values of parameters for determining the MRI pulse sequence. As a MRI pulse sequence is determined, the pulse verifying apparatus 100 may display a pulse sequence diagram indicating the determined MRI pulse sequence.

The pulse verifying apparatus 100 may display error information regarding a MRI pulse sequence on a pulse sequence diagram. Error information regarding a MRI pulse sequence may include information regarding whether a MRI pulse with an error exists, calculated characteristic values of MRI pulses, critical values of characteristics of MRI pulses, time points of occurrences of errors, and parameters related to calculated characteristic values of MRI pulses.

FIG. 6B is a diagram showing a method that the pulse verifying apparatus 100 displays error information regarding a MRI pulse sequence, according to an embodiment.

Referring to FIG. 6B, the pulse verifying apparatus 100 may display whether powers of RF pulses included in a MRI pulse sequence exceed critical values.

Powers of RF pulses to be irradiated to a target object may be determined based on shapes, magnitudes, durations, and the overall generation frequency of RF pulses from among parameters for determining RF pulses. For example, as magnitude 620 of a RF pulse increases from 0.817 to 5.817, power of the RF pulse may increase. The pulse verifying apparatus 100 may calculate powers of RF pulses to be irradiated to a target object based on shapes, magnitudes, durations, and the overall generation frequency of RF pulses and, if calculates powers of the RF pulses exceed a critical value of power of RF pulses, may determine that an error occurred with respect to RF pulses.

When RF pulses are shown on the time axis as a single function, an area formed between the function related to the RF pulses and the time axis may correspond to powers of the RF pulses.

Therefore, if powers of RF pulses irradiated to a target object exceeds a critical value, the pulse verifying apparatus 100 displays an image 610 indicating an area formed between RF pulses displayed on the time axis and the time axis, thereby indicating that powers of the RF pulses irradiated to the target object exceeded the critical value of power of RF pulses.

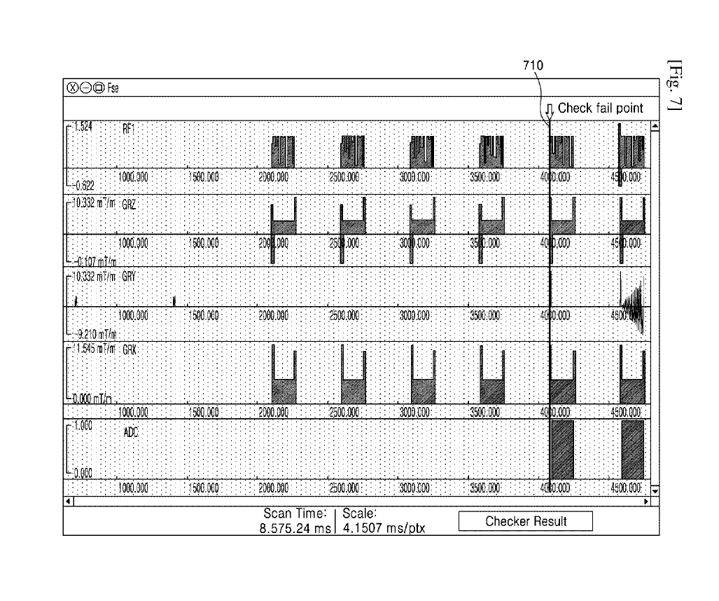

FIG. 7 is a diagram showing a method that the pulse verifying apparatus 100 outputs information regarding time points of occurrences of errors, according to an embodiment.

Referring to FIG. 7, the pulse verifying apparatus 100 may display a time point of occurrence of an error on a pulse sequence diagram.

The pulse verifying apparatus 100 may display MRI pulses on the time axis in a pulse sequence diagram according to time points of generation of MRI pulses.

The pulse verifying apparatus 100 may determine MRI pulses related to parameters with errors from among a plurality of MRI pulses constituting a MRI pulse sequence.

The pulse verifying apparatus 100 may determine location of a MRI pulse related to a parameter with an error at areas in a pulse sequence diagram. For example, the pulse verifying apparatus 100 may determine a time point of occurrence of a MRI pulse related to a parameter with an error based on parameters related to durations of MRI pulses and time points of generation of MRI pulses. As a time point of generation of a MRI pulse related to a parameter with an error is determined, the pulse verifying apparatus 100 may determine a location on the time axis corresponding to the determined time point.

As a location of a MRI pulse sequence related to a parameter with an error is determined, the pulse verifying apparatus 100 may display a pre-set image at the determined location.

For example, the pulse verifying apparatus 100 may determine a gradient magnetic field pulse corresponding to a set value of magnitude of the gradient magnetic field pulse exceeding a critical value of magnitude of the gradient magnetic field pulse, from among Y-axis gradient magnetic field pulses. Next, the pulse verifying apparatus 100 may determine a time period of the determined gradient magnetic field pulse in a pulse sequence diagram. Next, the pulse verifying apparatus 100 may display a pre-set image at an area corresponding to the time period, in the pulse sequence diagram. For example, the pulse verifying apparatus 100 may display a straight line 710 crossing the time axis at a location of the time axis in a pulse sequence diagram, the location corresponding to a time point of occurrence of an error.

Therefore, the pulse verifying apparatus 100 may indicate a location on the time axis in a pulse sequence diagram corresponding to a time point of occurrence of an error by displaying a pre-set image at the location.

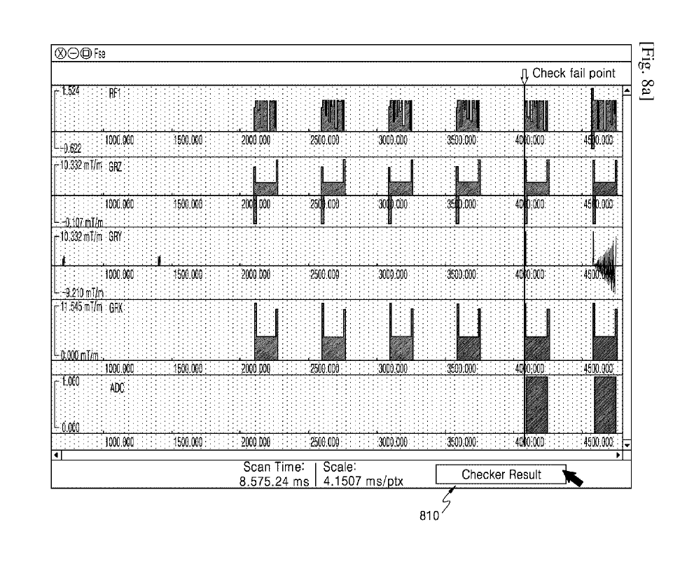

FIGS. 8A and 8B are diagrams showing a method that the pulse verifying apparatus 100 outputs error information regarding parameters, according to an embodiment.

Referring to FIG. 8A, the pulse verifying apparatus 100 may display error information according to an user input.

As an error occurs at a parameter, the pulse verifying apparatus 100 may display a user interface 810 for displaying error information. The user interface 810 for displaying error information may include a button interface.

If no error occurs at parameters, the pulse verifying apparatus 100 may not display the user interface 810 for displaying error information.

Referring to FIG. 8B, the pulse verifying apparatus 100 may display error information regarding parameters on a pulse sequence diagram.

As a user input for selecting the user interface 810 for displaying error information is received, the pulse verifying apparatus 100 may display a confirmation window 820 to indicate error information on a pulse sequence diagram. Error information may include a cause of an error 830, a time point 840 of occurrence of an error, and a user interface 850 for correcting an error.

For example, if a set value of the maximum magnitude of a Y-axis gradient magnetic field pulse exceed a critical value of the maximum magnitude of a Y-axis gradient magnetic field pulse, the pulse verifying apparatus 100 may display a description showing that the cause of an error 830 is the maximum magnitude of a gradient magnetic field pulse, a time point 840 at which a gradient magnetic field pulse corresponding to the set value of the maximum magnitude exceeding the critical value of the maximum magnitude is generated by a MRI apparatus, a user interface 850 for informing and correcting the set value of the maximum magnitude of the gradient magnetic field pulse, and a confirmation button 860 on the confirmation window 820.

Furthermore, as a user input for clicking the confirmation button 860 is received, the pulse verifying apparatus 100 may delete the confirmation window and re-display a pulse sequence diagram.

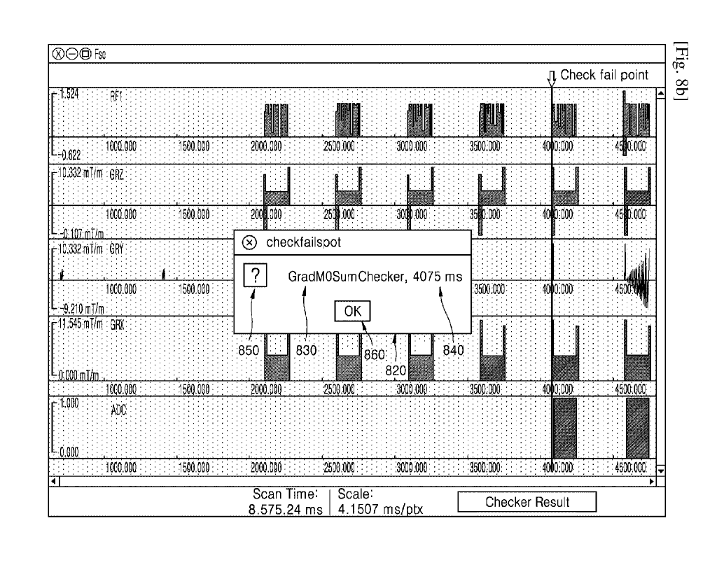

FIG. 9 is a method that the pulse verifying apparatus 100 displays error information regarding a MRI pulse sequence, according to an embodiment.

Referring to FIG. 9, the pulse verifying apparatus 100 may display error information on a pulse sequence diagram.

The pulse verifying apparatus 100 may indicate a specific cause of an error in numbers. For example, if a set value of the maximum magnitude of a Y-axis gradient magnetic field pulse is 7.597168 mT/m and a critical value of the maximum magnitude of a Y-axis gradient magnetic field pulse is 7 mT/m, the pulse verifying apparatus 100 may display a confirmation window 910 that shows the set value 920 of the maximum magnitude of the Y-axis gradient magnetic field pulse, the critical value 930 of the maximum magnitude of the Y-axis gradient magnetic field pulse, and information showing that the set value 920 of the maximum magnitude of the Y-axis gradient magnetic field pulse is equal to or greater than the critical value 930 of the maximum magnitude of the Y-axis gradient magnetic field pulse.

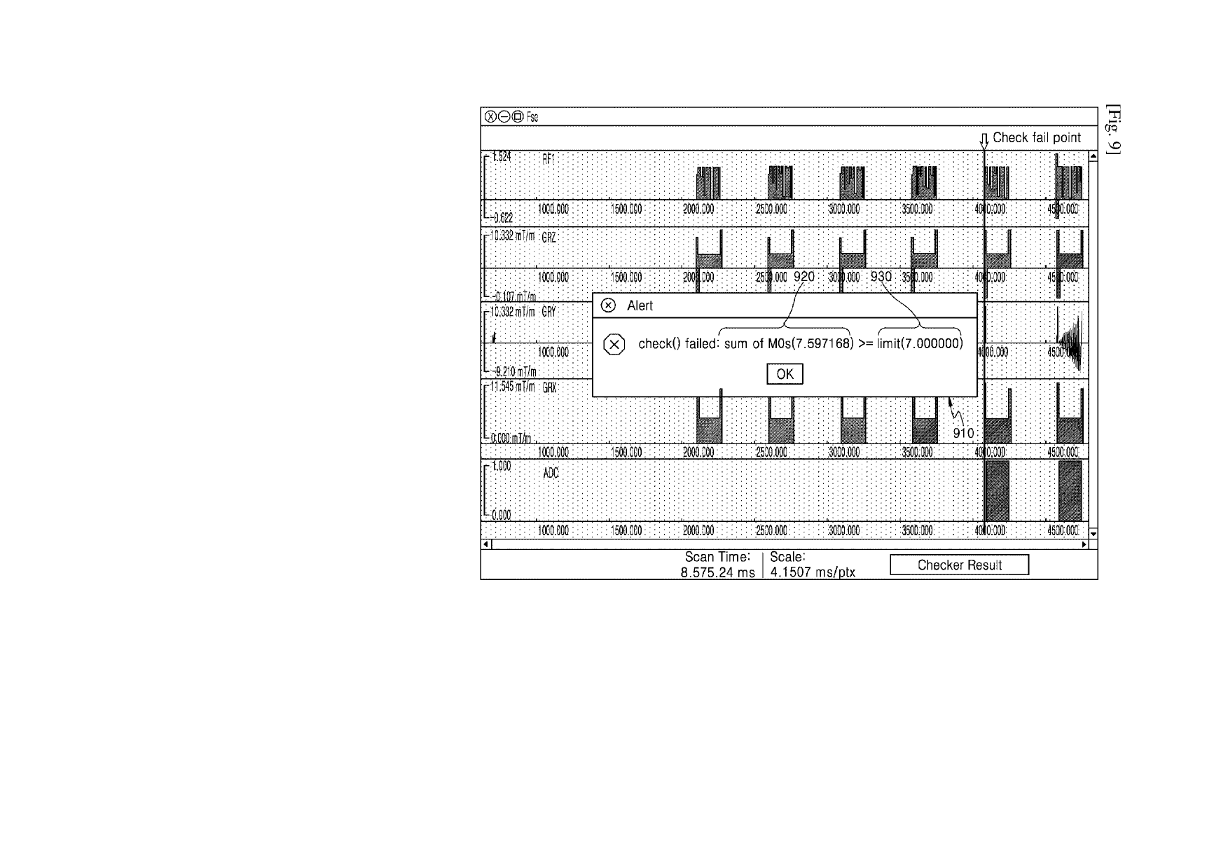

FIG. 10 is a diagram showing a method that the pulse verifying apparatus 100 displays a set value of a parameter with an error, according to an embodiment.

Referring to FIG. 10, the pulse verifying apparatus 100 may display a code related to a set value of a parameter with an error on a pulse sequence diagram.

As a user input for selecting the user interface 850 for informing a set value of a parameter with an error as shown in FIG. 8 is received, the pulse verifying apparatus 100 may display a code related to a set value of a parameter with an error on a pulse sequence diagram.

Furthermore, when a cursor is located within a pre-set distance from an image 710, which indicates a time point of occurrence of an error and is displayed in a pulse sequence diagram, the pulse verifying apparatus 100 may display a code related to a set value of a parameter with an error on the pulse sequence diagram.

A code related to a set value of a parameter may be a code regarding a parameter that is written for generating a MRI pulse sequence by a pulse sequence developer in the form of program codes. The pulse verifying apparatus 100 may receive a user input for inputting parameters of a MRI pulse sequence in the form of codes. Furthermore, the pulse verifying apparatus 100 may receive a file having recorded therein parameters of a MRI pulse sequence in the form of codes from an external device.

The pulse verifying apparatus 100 may display a code 1020 for setting a set value of a parameter with an error from among codes indicating parameters of a MRI pulse sequence. For example, if a set value of power of a RF pulse exceeds a critical value of power of a RF pulse, the pulse verifying apparatus 100 may display the code 1020 for setting a shape and a power of a RF pulse.

In this case, the pulse verifying apparatus 100 may display the code 1020 for setting a parameter with an error on an edit window 1010 for modifying codes.

If a user input for modifying a code for setting a set value of a parameter and a user input for clicking a confirmation button 1030 are received via an edit window, the pulse verifying apparatus 100 may modify an existing MRI pulse sequence based on the modified parameter.

As an existing MRI pulse sequence is modified, the pulse verifying apparatus 100 may display a pulse sequence diagram showing the modified MRI pulse sequence. Furthermore, the pulse verifying apparatus 100 may determine whether a modified set value of a parameter exceeds a critical value of the parameter again. Furthermore, based on the modified set value of the parameter, the pulse verifying apparatus 100 may determine whether a characteristic value of the MRI pulse sequence exceeds a critical value of a characteristic of the MRI pulse sequence again.

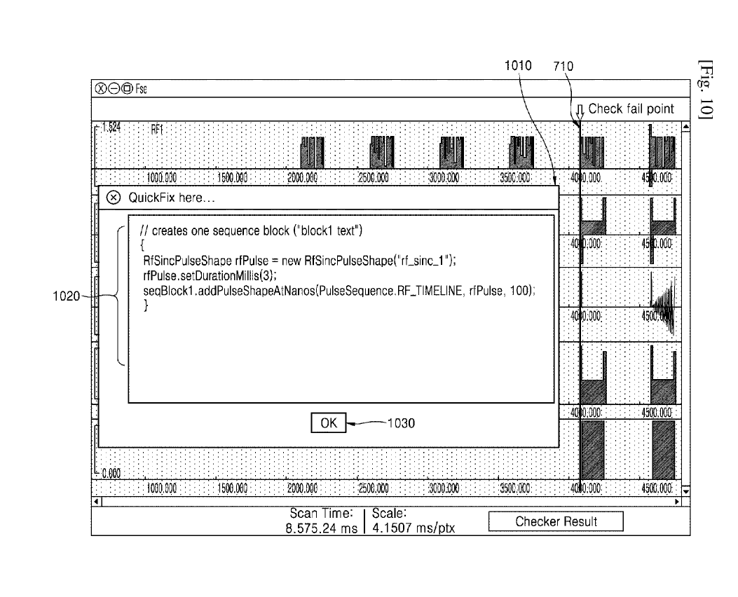

FIG. 11 is a diagram for describing a method that the pulse verifying apparatus 100 uses to obtain a critical value of a parameter, according to an embodiment.

Referring to FIG. 11, the pulse verifying apparatus 100 may obtain the critical value of the parameter based on a MRI apparatus or a target object.

Therefore, a permissible slew rate of a gradient magnetic field pulse or a permissible magnitude of a gradient magnetic field pulse may be determined based on the MRI apparatus. For example, the permissible slew rate of the gradient magnetic field pulse or the permissible magnitude of the gradient magnetic field pulse of the MRI apparatus may be determined based on a specification of the MRI apparatus or hardware configuration of the MRI apparatus.

Furthermore, a permissible magnitude of gradient magnetic field pulses, a permissible slew rate of the gradient magnetic field pulses, a permissible magnitude of RF pulses, and a permissible power of the RF pulses may be determined based on a target object. For example, under a same gradient magnetic field, nerve stimulation induced at an adult may be different from nerve stimulation induced at an infant. Furthermore, when same RF pulses are applied, RF power absorbed by a person having a relatively large body weight may be different from RF power absorbed by a person having a relatively small body weight.

The pulse verifying apparatus 100 may store critical values of parameters in correspondence to specifications, hardware configurations, and identifications of MRI apparatuses. Furthermore, the pulse verifying apparatus 100 may store critical values of parameters in correspondence to ages, body weights, and imaged body portions, and identifications of target objects.

The pulse verifying apparatus 100 may provide a user interface for setting critical values of parameters. For example, as a MRI apparatus or a target object is selected, the pulse verifying apparatus 100 may provide a user interface for setting critical values of parameters.

The user interface for setting critical values of parameters may include a user interface 1110 for setting a static magnetic field and a user interface 1120 for setting an identification number of a MRI apparatus.

As a user input for setting a static magnetic field is received via the user interface 1110 for setting a static magnetic field, the pulse verifying apparatus 100 may set a maximum magnitude value of a gradient magnetic field pulse corresponding to the set static magnetic field as a critical value of the maximum magnitude of a gradient magnetic field pulse. Furthermore, the pulse verifying apparatus 100 may set a slew rate value of a gradient magnetic field pulse corresponding to the set static magnetic field as a critical value of slew rate of a gradient magnetic field pulse.

For example, as the static magnetic field is set to 1.5 T, the pulse verifying apparatus 100 may set the maximum value of magnitude of a gradient magnetic field pulse corresponding to 1.5 T, which is 33 mT/m, as the critical value of the maximum magnitude of a gradient magnetic field pulse.

Furthermore, as a user input for setting an identification number of a MRI apparatus is received, the pulse verifying apparatus 100 may set a value of a parameter, which is stored in advance in correspondence to the identification number of the MRI apparatus, as a critical value of the parameter. Furthermore, the pulse verifying apparatus 100 may provide a user interface for selecting specification of a MRI apparatus or an identification of the MRI apparatus as the standard for obtaining a critical value.

Furthermore, a user interface for setting a critical value of a parameter may include user interfaces 1130 and 1140 for setting body weight and age of a target object, respectively. Furthermore, a user interface for setting a critical value of a parameter may include a user interface 1150 for selecting an imaged body portion to obtain a MR image or a user interface 1160 for setting identification information regarding a target object.

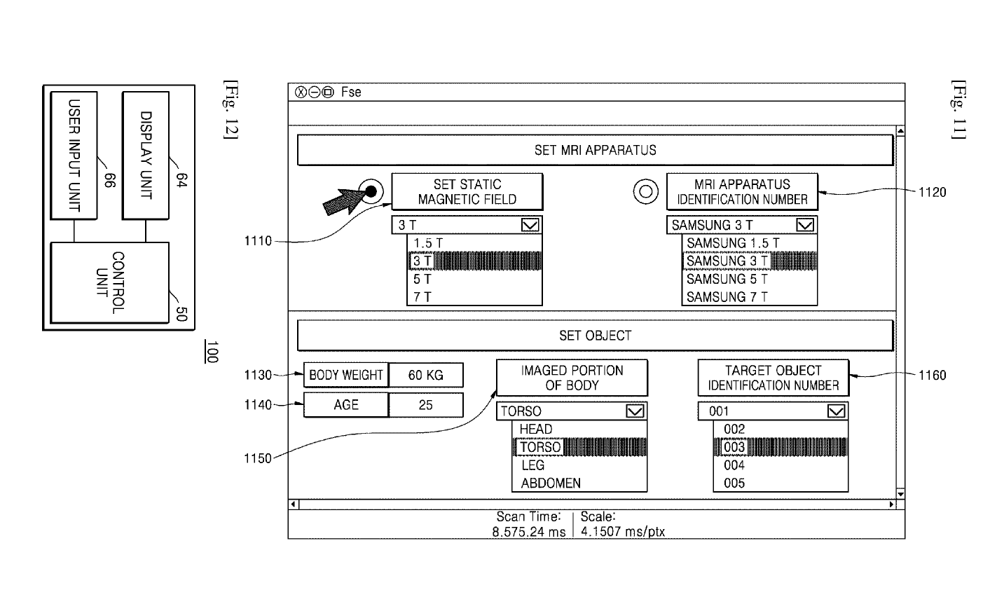

FIG. 12 is a block diagram showing the pulse verifying apparatus 100 according to an embodiment.

Referring to FIG. 12, the pulse verifying apparatus 100 may include a display unit 64, a user input unit 66, and a control unit 50. However, not all of the components shown in FIG. 12 are necessary components. The pulse verifying apparatus 100 may be embodied with more or less components than those shown in FIG. 12.

The pulse verifying apparatus 100 may be an MRI apparatus for picking up a MR image. Furthermore, the pulse verifying apparatus 100 may be a PACS viewer, a smart phone, a laptop computer, a personal digital assistant (PDA), or a tablet PC, but is not limited thereto.

The user input unit 66 may receive various user inputs for verifying a MRI pulse sequence. Furthermore, the user input unit 66 may receive various user inputs for operating a displayed user interface.

Furthermore, the user input unit 66 may receive at least one of an identification of a MRI apparatus to generate a plurality of MRI pulses according to a MRI pulse sequence and an identification of a target object to apply the plurality of MRI pulses.

Furthermore, the user input unit 66 may obtain a set value of a parameter for determining a MRI pulse sequence. Furthermore, the user input unit 66 may receive a user input for modifying a set value of a parameter via an edit window for modifying a set value of a parameter.

The control unit 50 may control components in the pulse verifying apparatus 100. The control unit 50 may control the user input unit 66 and the display unit 64.

Furthermore, the control unit 50 may compare a set value of a parameter to a critical value of the parameter and determine whether an error occurred with respect to the parameter based on a result of the comparison.

Furthermore, the control unit 50 may generate a MRI pulse sequence based on a set value of a parameter.

Furthermore, the control unit 50 may obtain a critical value of a parameter based on at least one of an identification of a MRI apparatus and an identification of a target object and compare a set value of the parameter to the obtained critical value of the parameter.

Furthermore, the control unit 50 may determine a MRI pulse related to a parameter corresponding to a set value exceeding a critical value from among a plurality of MRI pulses constituting a MRI pulse sequence and determine a location on the time axis in a pulse sequence diagram where the determined MRI pulse is displayed.

Furthermore, the control unit 50 may determine whether power of a RF pulse included in a MRI pulse sequence exceeds a pre-set critical value.

Furthermore, the control unit 50 may determine a location in a pulse sequence diagram corresponding to the critical value.

Furthermore, the control unit 50 may modify a MRI pulse sequence based on a modified set value of a parameter.

The display unit 64 may display various information for verifying a MRI pulse.

Furthermore, the display unit 64 may display a MRI pulse sequence, a pulse sequence diagram, and error information.

Furthermore, if an error occurs with respect to a parameter, the display unit 64 may display information regarding the error on a pulse sequence diagram corresponding to a MRI pulse sequence generated based on a set value of the parameter.

Furthermore, if power of a RF pulse exceeds a pre-set critical value, the display unit 64 may display an image indicating an area formed between the RF pulse displayed on the time axis in the pulse sequence diagram and the time axis.

Furthermore, the display unit 64 may display information regarding an error by changing a color of an area in which a pulse sequence diagram is displayed.

Furthermore, the display unit 64 may display information regarding a parameter corresponding to a set value exceeding a critical value.

Furthermore, the display unit 64 may display an edit window for modifying a set value of a parameter.

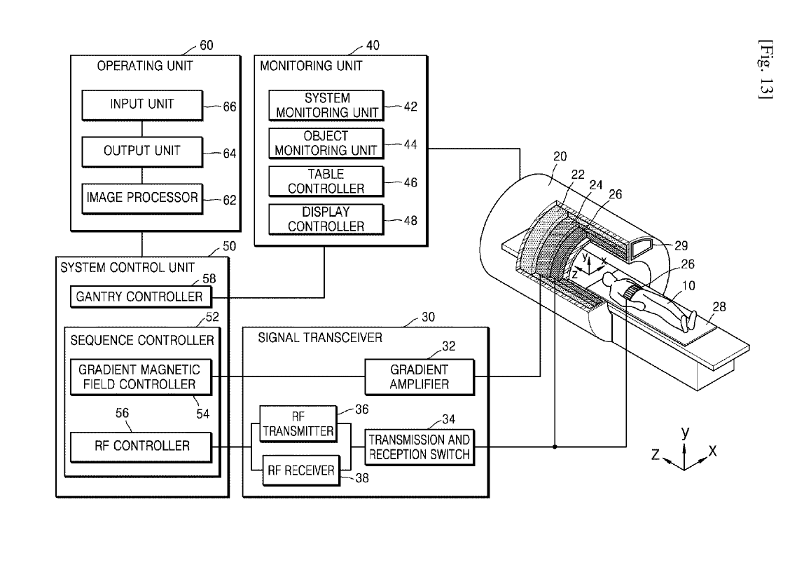

FIG. 13 is a block diagram showing the pulse verifying apparatus 100 according to another embodiment.

Referring to FIG. 13, the pulse verifying apparatus 100 may include a gantry 20, a signal transceiver 30, a monitoring unit 40, a control unit 50, and an operating unit 60.

The gantry 20 prevents external emission of electromagnetic waves generated by a main magnet 22, a gradient coil 24, and an RF coil 26. A magnetostatic field and a gradient magnetic field are formed in a bore in the gantry 20, and an RF signal is emitted toward an object 10.

The main magnet 22, the gradient coil 24, and the RF coil 26 may be arranged in a predetermined direction of the gantry 20. The predetermined direction may be a coaxial cylinder direction. The object 10 may be disposed on a table 28 that is capable of being inserted into a cylinder along a horizontal axis of the cylinder.

The main magnet 22 generates a magnetostatic field or a static magnetic field for aligning magnetic dipole moments of atomic nuclei of the object 10 in a constant direction. A precise and accurate MR image of the object 10 may be obtained due to a magnetic field generated by the main magnet 22 being strong and uniform.

The gradient coil 24 includes X, Y, and Z coils for generating gradient magnetic fields in X-, Y-, and Z-axis directions crossing each other at right angles. The gradient coil 24 may provide location information of each region of the object 10 by differently inducing resonance frequencies according to the regions of the object 10.

The RF coil 26 may emit an RF signal toward a patient and receive an MR signal emitted from the patient. In detail, the RF coil 26 may transmit, toward atomic nuclei and having precessional motion, an RF signal having the same frequency as that of the precessional motion to the patient, stop transmitting the RF signal, and then receive an MR signal emitted from the patient.

For example, in order to transit an atomic nucleus from a low energy state to a high energy state, the RF coil 26 may generate and apply an electromagnetic wave signal that is an RF signal corresponding to a type of the atomic nucleus, to the object 10. When the electromagnetic wave signal generated by the RF coil 26 is applied to the atomic nucleus, the atomic nucleus may transit from the low energy state to the high energy state. Then, when electromagnetic waves generated by the RF coil 26 disappear, the atomic nucleus to which the electromagnetic waves were applied transits from the high energy state to the low energy state, thereby emitting electromagnetic waves having a Lamor frequency. In other words, when the applying of the electromagnetic wave signal to the atomic nucleus is stopped, an energy level of the atomic nucleus is changed from a high energy level to a low energy level, and thus the atomic nucleus may emit electromagnetic waves having a Lamor frequency. The RF coil 26 may receive electromagnetic wave signals from atomic nuclei included in the object 10.

The RF coil 26 may be realized as one RF transmitting and receiving coil having both a function of generating electromagnetic waves each having an RF that corresponds to a type of an atomic nucleus and a function of receiving electromagnetic waves emitted from an atomic nucleus. Alternatively, the RF coil 26 may be realized as a transmission RF coil having a function of generating electromagnetic waves each having an RF that corresponds to a type of an atomic nucleus, and a reception RF coil having a function of receiving electromagnetic waves emitted from an atomic nucleus.

The RF coil 26 may be fixed to the gantry 20 or may be detachable. When the RF coil 26 is detachable, the RF coil 26 may be an RF coil for a part of the object, such as a head RF coil, a chest RF coil, a leg RF coil, a neck RF coil, a shoulder RF coil, a wrist RF coil, or an ankle RF coil.

The RF coil 26 may communicate with an external apparatus via wires and/or wirelessly, and may also perform dual tune communication according to a communication frequency band.

The RF coil 26 may communicate with an external apparatus via wires and/or wirelessly, and may also perform dual tune communication according to a communication frequency band.

The RF coil 26 may be a transmission exclusive coil, a reception exclusive coil, or a transmission and reception coil according to methods of transmitting and receiving an RF signal.

The RF coil 26 may be an RF coil having various numbers of channels, such as 16 channels, 32 channels, 72 channels, and 144 channels.

The gantry 20 may further include a display 29 disposed outside the gantry 20 and a display (not shown) disposed inside the gantry 20. The gantry 20 may provide predetermined information to the user or the object 10 through the display 29 and the display respectively disposed outside and inside the gantry 20.

The signal transceiver 30 may control the gradient magnetic field formed inside the gantry 20, i.e., in the bore, according to a predetermined MR sequence, and control transmission and reception of an RF signal and an MR signal.

The signal transceiver 30 may include a gradient amplifier 32, a transmission and reception switch 34, an RF transmitter 36, and an RF receiver 38.

The gradient amplifier 32 drives the gradient coil 24 included in the gantry 20, and may supply a pulse signal for generating a gradient magnetic field to the gradient coil 24 under the control of a gradient magnetic field controller 54. By controlling the pulse signal supplied from the gradient amplifier 32 to the gradient coil 24, gradient magnetic fields in X-, Y-, and Z-axis directions may be synthesized.

The RF transmitter 36 and the RF receiver 38 may drive the RF coil 26. The RF transmitter 36 may supply an RF pulse in a Lamor frequency to the RF coil 26, and the RF receiver 38 may receive an MR signal received by the RF coil 26.

The transmission and reception switch 34 may adjust transmitting and receiving directions of the RF signal and the MR signal. For example, the transmission and reception switch 34 may emit the RF signal toward the object 10 through the RF coil 26 during a transmission mode, and receive the MR signal from the object 10 through the RF coil 26 during a reception mode. The transmission and reception switch 34 may be controlled by a control signal output by an RF controller 56.

The monitoring unit 40 may monitor or control the gantry 20 or devices mounted on the gantry 20. The monitoring unit 40 may include a system monitoring unit 42, an object monitoring unit 44, a table controller 46, and a display controller 48.

The system monitoring unit 42 may monitor and control a state of the magnetostatic field, a state of the gradient magnetic field, a state of the RF signal, a state of the RF coil 26, a state of the table 28, a state of a device measuring body information of the object 10, a power supply state, a state of a thermal exchanger, and a state of a compressor.

The object monitoring unit 44 monitors a state of the object 10. In detail, the object monitoring unit 44 may include a camera for observing a movement or position of the object 10, a respiration measurer for measuring the respiration of the object 10, an electrocardiogram (ECG) measurer for measuring the electrical activity of the object 10, or a temperature measurer for measuring a temperature of the object 10.

The table controller 46 controls a movement of the table 28 where the object 10 is positioned. The table controller 46 may control the movement of the table 28 according to a sequence control of a sequence controller 52. For example, during moving imaging of the object 10, the table controller 46 may continuously or discontinuously move the table 28 according to the sequence control of the sequence controller 52, and thus the object 10 may be photographed in a field of view (FOV) larger than that of the gantry 20.

The display controller 48 controls the display 29 disposed outside the gantry 20 and the display disposed inside the gantry 20. In detail, the display controller 48 may control the display 29 and the display to be on or off, and may control a screen image to be output on the display 29 and the display. Also, when a speaker is located inside or outside the gantry 20, the display controller 48 may control the speaker to be on or off, or may control sound to be output via the speaker.

A control unit 50 may include the sequence controller 52 for controlling a sequence of signals formed in the gantry 20, and a gantry controller 58 for controlling the gantry 20 and the devices mounted on the gantry 20.

The sequence controller 52 may include the gradient magnetic field controller 54 for controlling the gradient amplifier 32, and the RF controller 56 for controlling the RF transmitter 36, the RF receiver 38, and the transmission and reception switch 34. The sequence controller 52 may control the gradient amplifier 32, the RF transmitter 36, the RF receiver 38, and the transmission and reception switch 34 according to a pulse sequence received from the operating unit 60. Here, the pulse sequence includes all information required to control the gradient amplifier 32, the RF transmitter 36, the RF receiver 38, and the transmission and reception switch 34. For example, the pulse sequence may include information about a strength, an application time, and application timing of a pulse signal applied to the gradient coil 24.

The operating unit 60 may transmit pulse sequence information to the control unit 50 and control the operations of the entire pulse verifying apparatus 100.

The operating unit 60 may include an image processor 62 for processing a MR signal received from the RF receiver 38, a display unit 64, and a user input unit 66.

The image processor 62 may process the MR signal received from the RF receiver 38 so as to generate MR image data of the object 10.

The image processor 62 performs any one of various signal processes, such as amplification, frequency transformation, phase detection, low frequency amplification, and filtering, on an MR signal received by the RF receiver 38.

The image processor 62 may arrange digital data in a k space (also referred to as a Fourier space or a frequency space) of a memory, and rearrange the digital data into image data by performing 2D or 3D Fourier transformation.

The image processor 62 may perform a composition process or a difference calculation process on image data if required. The composition process may include performing an addition process on a pixel or a maximum intensity projection (MIP) process. The image processor 62 may store not only the rearranged image data but also image data on which a composition process or a difference calculation process is performed, in a memory (not shown) or an external server.

The image processor 62 may perform any of the signal processes on the MR signal in parallel. For example, the image processor 62 may perform a signal process on a plurality of MR signals received by a multi-channel RF coil in parallel so as to rearrange the plurality of MR signals into image data.

The output unit 64 may output image data generated or rearranged by the image processor 62 to the user. Also, the output unit 64 may output information required for the user to manipulate the MRI system, such as user interface (UI), user information, or object information. The output unit 64 may include a speaker, a printer, a cathode-ray tube (CRT) display, a liquid crystal display (LCD), a plasma display panel (PDP), an organic light-emitting device (OLED) display, a field emission display (FED), a light-emitting diode (LED) display, a vacuum fluorescent display (VFD), a digital light processing (DLP) display, a PFD display, a 3-dimensional (3D) display, or a transparent display, or any one of various output devices that are well known to one of ordinary skill in the art.

A user may input object information, parameter information, a scan condition, a pulse sequence, or information about image composition or difference calculation by using the user input unit 66. The user input unit 66 may include a keyboard, a mouse, a track ball, a voice recognizer, a gesture recognizer, or a touch screen, or may include any one of other various input devices that are well known to one of ordinary skill in the art.

The operating unit 60 requests the system control unit 50 to transmit pulse sequence information while controlling an overall operation of the MRI system.

The operating unit 60 may include an image processor 62 for processing an MR signal received from the RF receiver 38, an output unit 64, and an input unit 66.

The image processor 62 processes an MR signal received from the RF receiver 38 so as to generate MR image data of the object 10.

The image processor 62 performs any one of various signal processes, such as amplification, frequency transformation, phase detection, low frequency amplification, and filtering, on an MR signal received by the RF receiver 38.

The image processor 62 may arrange digital data in a k space (for example, also referred to as a Fourier space or frequency space) of a memory, and rearrange the digital data into image data via 2D or 3D Fourier transformation.

The image processor 62 may perform a composition process or difference calculation process on image data if required. The composition process may include an addition process on a pixel or a maximum intensity projection (MIP) process. The image processor 62 may store not only rearranged image data but also image data on which a composition process or difference calculation process is performed, in a memory (not shown) or an external server.

Signal processes applied to MR signals by the image processor 62 may be performed in parallel. For example, a signal process may be performed on a plurality of MR signals received by a multi-channel RF coil in parallel so as to rearrange the plurality of MR signals as image data.

The output unit 64 may output image data generated or rearranged by the image processor 62 to the user. Also, the output unit 64 may output information required for the user to manipulate the MRI system, such as user interface (UI), user information, or object information. The output unit 64 may include a speaker, a printer, a cathode-ray tube (CRT) display, a liquid crystal display (LCD), a plasma display panel (PDP), an organic light-emitting device (OLED) display, a field emission display (FED), a light-emitting diode (LED) display, a vacuum fluorescent display (VFD), a digital light processing (DLP) display, a PFD display, a 3-dimensional (3D) display, or a transparent display, or any one of various output devices that are well known to one of ordinary skill in the art.

The user may input object information, parameter information, a scan condition, a pulse sequence, or information about image composition or difference calculation by using the input unit 66. The input unit 66 may include a keyboard, a mouse, a track ball, a voice recognizer, a gesture recognizer, or a touch screen, or may include any one of other various input devices that are well known to one of ordinary skill in the art.

The signal transceiver 30, the monitoring unit 40, the system control unit 50, and the operating unit 60 are separate components in FIG. 13, but it is obvious to one of ordinary skill in the art that functions of the signal transceiver 30, the monitoring unit 40, the system control unit 50, and the operating unit 60 may be performed by another component.

For example, the image processor 62 converts the MR signal received from the RF receiver 38 into a digital signal in FIG. 1, but alternatively, the conversion of the MR signal into the digital signal may be performed by the RF receiver 38 or the RF coil 26.

The gantry 20, the RF coil 26, the signal transceiver 30, the monitoring unit 40, the system control unit 50, and the operating unit 60 may be connected to each other via wires or wirelessly, and when they are connected wirelessly, the MRI system may further include an apparatus (not shown) for synchronizing clocks therebetween. Communication between the gantry 20, the RF coil 26, the signal transceiver 30, the monitoring unit 40, the system control unit 50, and the operating unit 60 may be performed by using a high-speed digital interface, such as low voltage differential signaling (LVDS), asynchronous serial communication, such as universal asynchronous receiver transmitter (UART), a low-delay network protocol, such as an error synchronous serial communication or controller area network (CAN), or optical communication, or any other communication method that is well known to one of ordinary skill in the art.

The exemplary embodiment of the present invention can be implemented in the form of a recording medium that includes computer executable instructions, such as program modules, being executed by a computer. Computer-readable media can be any available media that can be accessed by the computer and includes both volatile and nonvolatile media, removable and non-removable media. In addition, the computer-readable media may include computer storage media and communication media. Computer storage media includes both the volatile and non-volatile, removable and non-removable media implemented as any method or technology for storage of information such as computer readable instructions, data structures, program modules, or other data. The medium of communication is typically computer-readable instructions, and other data in a modulated data signal such as data structures, program modules, or carrier, or other transport mechanism and includes any information delivery media.

The foregoing description is for illustrative purposes, and one of ordinary skill in the art will understand that the embodiments described above may be easily transformed into other specific forms without changing the technical spirit or essential features of the inventive concept. Therefore, the embodiments described above are merely examples and are not for purposes of limitation. For example, each component described as a single component may be embodied as distributed components, and components described as distributed components may also be practiced in a combined form.

The scope of the inventive concept is defined not by the detailed description of the inventive concept but by the appended claims, and all differences within the scope will be construed as being included in the inventive concept.

1. A pulse verifying apparatus comprising:
a user input unit, which obtains a set value of a parameter for determining a magnetic resonance imaging (MRI) pulse sequence;
a control unit, which compares the set value of the parameter to a critical value of the parameter and, based on a result of the comparison, determines whether an error occurred with respect to the parameter; and
a display unit, which, if an error occurred with respect to the parameter, displays information regarding the error on a pulse sequence diagram corresponding to a MRI pulse sequence generated based on the set value of the parameter.

2. The pulse verifying apparatus of claim 1, wherein the user input unit receives at least one of an identification of a MRI apparatus to generate a plurality of MRI pulses according to the MRI pulse sequence and an identification of a target object to which to apply the plurality of MRI pulses, and
the control unit obtains the critical value of the parameter based on at least one of the identification of the MRI apparatus and the identification of the target object and compares the set value of the parameter to the obtained critical value of the parameter.

3. The pulse verifying apparatus of claim 1, wherein the MRI pulse sequence comprises a radio frequency (RF) pulse and a gradient magnetic field pulse, and
the parameter comprises at least one of a slew rate of the gradient magnetic field pulse, a magnitude of the gradient magnetic field pulse, and a magnitude of the RF pulse.

4. The pulse verifying apparatus of claim 1, wherein the control unit generates the MRI pulse sequence based on the set value of the parameter, and
the display unit displays the pulse sequence diagram indicating the generated MRI pulse sequence.

5. The pulse verifying apparatus of claim 1, wherein the control unit determines a MRI pulse related to a parameter of which a set value exceeds a critical value corresponding to the parameter from among a plurality of MRI pulses and determines a location on a time axis in the pulse sequence diagram where the determined MRI pulse sequence is displayed, and
the display unit displays a pre-set image on the determined location.

6. The pulse verifying apparatus of claim 1, wherein the control unit determines whether power of a RF pulse included in the MRI pulse sequence exceeds a pre-set critical value, and,
if the power of the RF pulse exceeds the pre-set critical value, the display unit displays an image indicating an area formed between the RF pulse in the pulse sequence diagram and a time axis in the pulse sequence diagram.

7. The pulse verifying apparatus of claim 1, wherein the control unit determines a location in the pulse sequence diagram corresponding to the critical value, and
the display unit displays a pre-set image at the determined location.

8. The pulse verifying apparatus of claim 1, wherein the display unit displays the information regarding the error by changing a color of an area in which the pulse sequence diagram is displayed.

9. The pulse verifying apparatus of claim 1, wherein the display unit displays information regarding a parameter of which a set value exceeds a critical value corresponding to the parameter.

10. The pulse verifying apparatus of claim 9, wherein the display unit displays an edit window for modifying the set value of the parameter,
the user input unit receives a user input for modifying the set value of the parameter via the edit window, and
the control unit modifies the MRI pulse sequence based on the modified set value.

11. A method of verifying a pulse sequence, the method comprising:
obtaining a set value of a parameter for determining a MRI pulse sequence;
comparing the set value of the parameter to a critical value of the parameter;
based on a result of the comparison, determining whether an error occurred with respect to the parameter; and,
if an error occurred with respect to the parameter, displaying information regarding the error on a pulse sequence diagram corresponding to a MRI pulse sequence generated based on the set value of the parameter.

12. The method of claim 11, wherein the comparing of the set value of the parameter to the critical value of the parameter comprises:
receiving at least one of an identification of a MRI apparatus to generate a plurality of MRI pulses according to the MRI pulse sequence and an identification of a target object to apply the plurality of MRI pulses, and
obtaining a critical value of the parameter based on at least one of the identification of the MRI apparatus and the identification of the target object, and
comparing the set value of the parameter to the obtained critical value of the parameter.

13. The method of claim 11, wherein the MRI pulse sequence comprises a RF pulse and a gradient magnetic field pulse, and
the parameter comprises at least one of a slew rate of the gradient magnetic field pulse, a magnitude of the gradient magnetic field pulse, and a magnitude of the RF pulse.

14. The method of claim 11, wherein the displaying of the information regarding the error on the pulse sequence diagram comprises:
generating the MRI pulse sequence based on the set value of the parameter; and
displaying the pulse sequence diagram indicating the generated MRI pulse sequence.

15. The method of claim 11, wherein the displaying of the information regarding the error on the pulse sequence diagram comprises:
determining a MRI pulse related to a parameter of which a set value exceeding a critical value corresponding to the parameter from among a plurality of MRI pulses;
determining a location on a time axis in the pulse sequence diagram where the determined MRI pulse sequence is displayed; and
displaying a pre-set image on the determined location.

16. The method of claim 11, further comprising determining whether power of a RF pulse included in the MRI pulse sequence exceeds a pre-set critical value,
wherein the displaying of the information regarding the error on the pulse sequence diagram comprises, if the power of the RF pulse exceeds the pre-set critical value, displaying an image indicating an area formed between the RF pulse in the pulse sequence diagram and a time axis in the pulse sequence diagram.

17. The method of claim 11, wherein the displaying of the information regarding the error on the pulse sequence diagram comprises:
determining a location in the pulse sequence diagram corresponding to the critical value; and
displaying a pre-set image at the determined location.

18. The method of claim 11, wherein the displaying of the information regarding the error on the pulse sequence diagram comprises displaying the information regarding the error by changing a color of an area in which the pulse sequence diagram is displayed.

19. The method of claim 11, wherein the displaying of the information regarding the error on the pulse sequence diagram comprises displaying information regarding a parameter of which a set value exceeds a critical value corresponding to the parameter.

20. The method of claim 19, wherein the displaying of the information related to the parameter comprises:
displaying an edit window for modifying the set value of the parameter, receiving a user input for modifying the set value of the parameter via the edit window, and
modifying the MRI pulse sequence based on the modified set value.
